(19) United States
(12) Patent Application Publication
(10) Pub. No.: US 2002/0187136 A1
(43) Pub. Date: Dec. 12, 2002

(54) Use of bacterial phage associated lysing enzymes for treating various illnesses

(76) Inventors: Lawrence Loomis, Columbia, MD (US); Vincent Fischetti, West Hempstead, NY (US)

Correspondence Address: HELLER EHRMAN WHITE & MCAULIFFE LLP, 1666 K STREET,NW, SUITE 300, WASHINGTON, DC, 20006, US

(21) Appl. No.: 09/844,435

(22) Filed: Apr. 30, 2001

Related U.S. Application Data
(63) Continuation-in-part of application No. 09/560,650, filed on Apr. 28, 2000.
(63) Continuation-in-part of application No. 09/752,732, filed on Jan. 3, 2001.

(51) Int. Cl. A61K 38/43

(52) U.S. Cl. 424/94.1

(57) ABSTRACT

A composition and method for treating bacterial infections is disclosed which comprises the treatment of an individual with an effective amount of at least one lytic enzyme produced by a bacteria infected with a bacteriophage specific for said bacteria wherein at least one lytic enzyme is selected from the group consisting of shuffled lytic enzymes, chimeric lytic enzymes, holin enzymes, and combinations thereof. A carrier may be used for delivering the lytic enzyme. This method, and composition can be used for the treatment of upper respiratory infections, skin infections, wounds, and burns, vaginal infections, eye infections, intestinal disorders and dental problems.

### BACKGROUND OF THE INVENTION

1. Field of the Invention

The present invention discloses methods and compositions for the treatment of bacterial infections by the use of lytic enzymes, modified lytic enzymes such as shuffled lytic enzymes, and chimeric lytic enzymes, and optionally, holin enzymes blended with an appropriate carrier suitable for the treatment of the infection.

2. Description of the Prior Art

In the past, antibiotics have been used to treat various infections. The work of Selman Waksman in the introduction and production of Streptomycetes, and Dr. Fleming's discovery of penicillin, as well as the work of numerous others in the field of antibiotics, are well known. Over the years, there have been additions and chemical modifications to the “basic” antibiotics in attempts to make them more powerful, or to treat people allergic to these antibiotics.

Additionally, others have found new uses for these antibiotics. U.S. Pat. No. 5,260,292 (Robinson et al.) discloses the topical treatment of acne with aminopenicillins. The method and composition for topically treating acne and acneiform dermal disorders includes applying an amount of an antibiotic selected from the group consisting of ampicillin, amoxicillin, other aminopenicillins, and cephalosporins, and derivatives and analogs thereof, effective to treat the acne and acneiform dermal disorders. U.S. Pat. No. 5,409,917 (Robinson et al.) discloses the topical treatment of acne with cephalosporins.

However, as more antibiotics have been prescribed or used at an ever increasing rate for a variety of illnesses, increasing numbers of bacteria have developed a resistance to antibiotics. Larger doses of stronger antibiotics are now being used to treat ever more resistant strains of bacteria. Multiple antibiotic resistant bacteria have consequently developed. The use of more antibiotics and the number of bacteria showing resistance has led to increasing the amount of time that the antibiotics need to be used. Broad, nonspecific antibiotics, some of which have detrimental effects on the patient, are now being used more frequently. Also, antibiotics do not easily penetrate mucus linings.

Additionally, the number of people allergic to antibiotics appears to be increasing. Consequently, other efforts have been sought to first identify and then kill bacteria.

Attempts have been made to treat bacterial diseases with the use of bacteriophages. U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which comprises introducing into the mouth bacteriophages parasitic to bacteria which possess the property of readily adhering to the salivary pellicle.

It is to be noted that the direct introduction of bacteriophages into an animal to prevent or fight diseases has certain drawbacks. Typically, the bacteria should be in the right growth phase for the phage to attach. Both the bacteria and the phage should be in the correct and synchronized growth cycles. Additionally, there should be the right number of phages to attach to the bacteria; if there are too many or too few phages, there will be either no attachment or no production of the lysing enzyme. The phage should also be active enough. The phages are also inhibited by many things including bacterial debris from the organism it is going to attack. Further complicating the direct use of bacteriophages to treat bacterial infections is the possibility of immunological reactions, rendering the phage nonfunctional.

Consequently, others have explored the use of safer and more effective means to treat and prevent bacterial infections.

U.S. Pat. No. 5,604,109 (Fischetti et al.) relates to the rapid detection of Group A streptococci in clinical specimens, through the enzymatic digestion by a semi-purified Group C streptococcal phage associated lysin enzyme. The present invention is based upon the discovery that phage associated lytic enzymes specific for bacteria infected with a specific phage can effectively and efficiently break down the cell wall of the bacterium in question. At the same time, in most if not all cases, the semipurified enzyme is lacking in mammalian cell receptors and therefore tends to be less destructive to mammalian proteins and tissues when present during the digestion of the bacterial cell wall.

U.S. Pat. No. 5,985,271 (Fischetti, et. al.), U.S. Pat. No. 5,997,862 (Fischetti et al.), and U.S. Pat. No. 6,017,528 (Fischetti et al.) disclose the compositions and their use in an oral delivery mode, such as a candy, chewing gum, lozenge, troche, tablet, a powder, an aerosol, a liquid or a liquid spray that contains a lysin enzyme produced by group C streptococcal bacteria infected with a C1 bacteriophage for the prophylactic and therapeutic treatment of Streptococcal A throat infections, commonly known as strep throat. This lysin enzyme is described in U.S. Pat. No. 5,604,109.

The same general technique used to produce and purify a lysin enzyme shown in U.S. Pat. No. 5,604,109 may be used to manufacture other lytic enzymes produced by bacteria infected with a bacteriophage specific for that bacteria. Depending on the bacteria, there may be variations in the growth media and conditions.

The use of phage associated lytic enzymes produced by the infection of a bacteria with a bacteria specific phage has numerous advantages for the treatment of diseases. As the phage are targeted for specific bacteria, the lytic enzymes generally do not interfere with normal flora. Also, lytic phages primarily attack cell wall structures, which are not affected by plasmid variation. The actions of the lytic enzymes are fast and do not depend on bacterial growth. Additionally, lytic enzymes can be directed to the mucosal lining, where, in residence, they will be able to kill colonizing bacteria.

U.S. Pat. No. 6,056,954 (Fischetti et al.) discloses a method and composition for the prophylactic and/or therapeutic treatment of bacterial infections, comprising administering an effective amount of at least one lytic enzyme produced by a bacteria infected with a bacteriophage specific for the bacteria to the site of the infection. The lytic enzyme preferably comprises a carrier suitable for delivering the lytic enzyme to the site of the infection. This method and treatment may be used for treating upper respiratory infections, topical infections, vaginal infections, eye infections, ear infections, for parenteral treatment, and for most other bacterial infections.

U.S. Pat. No. 6,056,955 (Fischetti et al.) discloses the topical treatment of streptococcal infections.

### SUMMARY OF THE INVENTION

Methods for obtaining and purifying the lytic enzyme produced by a bacterium infected with the bacteriophage are known. Some recent evidence suggests that the phage enzyme that lyses the Streptococcus organism may actually be a bacterial enzyme that is used to construct the cell wall and the phage. While replicating in the bacterium, a phage gene product may cause the upregulation or derepression of bacterial enzyme for the purpose of releasing the bacteriophage. These bacterial enzymes may be tightly regulated by the bacterial cell and are used by the bacteria for the construction and assembly of the cell wall.

The use of these lytic enzymes for the prophylactic and therapeutic treatment of bacterial diseases, however, has not been explored in a sufficient manner, except by the inventors of the present invention. The lytic enzymes produced by bacterial phages generally are specific and effective for killing select bacteria.

The present invention discloses the extraction and use of a variety of bacterial phage associated holin lytic enzymes, chimeric lytic enzymes, and shuffled lytic enzymes, in addition to lytic enzymes, for increased efficiency for the treatment of a wide variety of illnesses caused by bacterial infections. More specifically, the present invention provides a pharmaceutical composition comprising at least one bacteria-associated phage enzyme that is isolated from one or more bacteria species and includes phage lytic and/or holin enzymes. In one embodiment, the lytic or holin enzymes, including their isozymes, analogs, or variants, are used in a modified form. In another embodiment the lytic or holin enzymes, including their isozymes, analogs, or variants, are used in a combination of natural and modified forms. The modified forms of lytic and holin enzymes are made synthetically by chemical synthesis and/or DNA recombinant techniques. and, more preferably, the enzymes are made synthetically by chimerization and/or shuffling.

According to one embodiment, the pharmaceutical composition includes one or more natural lytic enzyme produced by the bacterial organism, after being infected with a particular bacteriophage, for prophylactic or therapeutic treatment. Preferably, the pharmaceutical composition contains combinations of one or more natural lytic enzyme and one or more chimeric or shuffled lytic enzymes.

Chimeric enzymes are enzymes which are a combination of two or more enzymes having two or more active sites such that the chimeric enzyme can act independently on the same or different molecules. This will allow for potentially treating two or more different bacterial infections at the same time.

Holin enzymes produce holes in the cell membrane. More specifically, holins form lethal membrane lesions that terminate respiration. Like the lytic enzymes, many holin enzymes are coded for and carried by a phage. In fact, it is quite common for the genetic code of the holin enzyme to be found next to or even within the code for the lytic enzyme in the phage. Most holin sequences are short, and overall, hydrophobic in nature, with a highly hydrophilic carboxy-terminal domain. In many cases, the putative holin is encoded on a different reading frame within the enzymatically active domain of the phage. In other cases, the holin is encoded on the DNA next or close to the DNA coding for the phage. The holin is frequently synthesized during the late stage of phage infection and found in the cytoplasmic membrane where it causes membrane lesions.

Holin enzymes can be grouped into two general classes based on primary structure analysis. Class I holins are usually 95 residues or longer and may have three potential transmembrane domains. Class II holins are usually smaller, at approximately 65-95 residues, and the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all of the phages. Holins have been shown to be present or suggested for among others, lactococcal bacteriophage Tuc2009, lactococcal □LC3, pneumococcal bacteriophage EJ-1, Lactobacillus gasseri bacteriophage □adh, Staphylococcus aureus bacteriophage Twort, Listeria monocytogenes bacteriophages, pneumococcal phage Cp-1, Bacillus subtillis phage □29, Lactobacillus delbrueckki bacteriophage LL-H lysin, and bacteriophage □11 of Staphylococcus aureus. (Loessner, et al., Journal of Bacteriology, August 1999, p. 4452-4460).

Shuffled enzymes are enzymes in which the genes, gene products, or peptides for more than one related phage enzyme have been randomly cleaved and reassembled into a more active or specific enzyme. Shuffled oligonucleotides, peptides or peptide fragment molecules are then selected or screened to identify a molecule having a desired functional property. This method is described, for example, in Stemmer, U.S. Pat. No. 6,132,970. (Method of shuffling polynucleotides); Kauffman, U.S. Pat. No. 5, 976,862 (Evolution via Condon-based Synthesis) and Huse, U.S. Pat. No. 5,808,022 (Direct Codon Synthesis). The contents of these patents are incorporated herein by reference.

Shuffling is used to create an enzyme 10 to 100 fold more active than the template. The template enzyme is selected among different varieties of lysin or holin enzymes. The shuffled enzyme constitutes, for example, one or more binding domains and one or more catalytic domains. Each of the binding or catalytic domains is derived from the same or different phage or phage enzyme. The shuffled domains are either oligonucleotide based molecules, as gene or gene products, that either alone or in combination with other genes or gene products are translatable into a peptide fragment, or they are peptide based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable of translation into a peptide.

All isozymes, variants or analogs of the bacterial-associated phage enzymes of the invention, whether natural or modified, are encompassed and included within the scope of the invention.

More specifically, the sequence of enzymes when purified can be determined by conventional techniques, and rearrangements of primary structures can be achieved by state of the art techniques, such as shuffling, to increase the activity and stability of the enzyme(s). Shuffling also allows for combination enzymes (“chimeric enzymes”) to have more than one activity.

The creation, purification, and isolation of chimeric, shuffled, and holin enzymes are well known to those skilled in the art. In particular, U.S. Pat. No. 6,132,970 (Stemmer) discloses a number of new techniques, and modifications of more established procedures, for the creation of these enzymes. The proposed invention utilizes these techniques and applies them for the enhancement of specifically noted phage associated lytic enzymes. The technique for isolating lysin enzymes found in U.S. Pat. No. 6,056,954 (also incorporated herein by reference) may be applied to other phage associated lytic enzymes. Similarly, other state of the art techniques may be used to isolate lytic enzymes.

In a preferred embodiment of the invention, shuffled enzymes are used to treat bacterial infections, thereby increasing the speed and efficiency with which the bacteria are killed.

Chimeric enzymes may also be used to treat one bacterial infection by cleaving the cell wall of the bacteria in more than one location.

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. Jul. 1,1998 164(1); 159-67.

In another experiment an active chimeric cell wall lytic enzyme (TSL) has been constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumococcal cell walls.

A preferred embodiment of this invention discloses the use of chimeric lytic enzymes to treat two infectious bacteria at the same time, or to cleave the cell wall of a bacteria in two different locations.

In another embodiment of the invention, holin enzymes are used in conjunction with the lytic enzymes to accelerate the speed and efficiency at which the bacteria are killed. Holin enzymes may also be in the form of chimeric and/or shuffled enzymes. Holin enzymes may also be used alone in the treatment of bacterial infections.

It is an object of the invention to use phage associated lytic enzymes in combination with chimeric or shuffled lytic enzymes to prophylactically and therapeutically treat bacterial diseases.

In another embodiment of the invention, chimeric lytic enzymes are used to prophylactically and therapeutically treat bacterial diseases.

In yet another embodiment of the invention, shuffled lytic enzymes are used to prophylactically and therapeutically treat bacterial infections.

In yet another embodiment of the invention, holin enzymes are used in conjunction with phage associated lytic enzymes to prophylactically and therapeutically treat bacterial infections.

In another embodiment of the invention, holin enzymes alone are used to prophylactically and therapeutically treat bacterial infections.

In another embodiment of the invention, the holin enzymes are shuffled holin enzymes or chimeric holin enzymes, in either combination with or independent of the lytic enzymes.

The invention (which incorporates U.S. Pat. No. 5,604,109 in its entirety by reference) uses a lytic enzyme produced by the bacterial organism after being infected with a particular bacteriophage as either a prophylactic treatment for preventing those who have been exposed to others who have the symptoms of an infection from getting sick, or as a therapeutic treatment for those who have already become ill from the infection. The present invention is based upon the discovery that phage lytic enzymes specific for bacteria infected with a specific phage can effectively and efficiently break down the cell wall of the bacterium in question. At the same time, the semipurified enzyme is lacking in proteolytic enzymatic activity and therefore nondestructive to mammalian proteins and tissues when present during the digestion of the bacterial cell wall. As discussed above, the lytic enzymes may be chimeric, shuffled or “natural,” and may be in combination with at least one holin enzyme, which may also be chimeric, shuffled, or “natural.”

In one embodiment of the invention, the prophylactic and therapeutic treatment of a variety of illnesses caused by Streptococcal pneumoniae, Streptococcus fasciae, and Hemophilus influenza are disclosed. In another embodiment of the invention, infections caused by Listeria, Salmonella, E. coli, and Campylobacter, are treated by the use of other shuffled and/or lytic enzymes, possibly in combination with holin and other lytic enzymes. The bacteria infecting the digestive system can be treated by incorporating the enzymes in suppository enemas, in syrups, or in other carriers to get directly to the site of the infection(s).

In another embodiment of the invention, lytic enzymes, modified lytic enzymes such as shuffled lytic enzymes and/or chimeric lytic enzymes are incorporated into bandages to prevent or treat infections of burns and wounds. In yet another embodiment of the invention, the lytic enzymes of phage associated with Staphylococcus or Pseudomonas are incorporated into bandages to prevent or treat infections of burns and wounds. Staphylococcus, Pseudomonas, and Streptococcus are frequently found in dermatologiical infections. Similarly, holin and other lytic enzymes may be used in combination with the chimeric and/or shuffled enzymes.

Vaginal infections caused by Group B Streptococcus can cause premature birth and subsequent complications resulting in neonatal sepsis. Chimeric lytic enzymes, shuffled lytic enzymes, lytic enzymes, alone or in combination with holin lytic enzymes and other lytic enzymes, incorporated into tampons specific for group B strep would prevent infection of the neonate during birth without disturbing normal vaginal flora so that women would not be overcome by yeast infection as a result of antibiotic therapy.

In another embodiment of the invention, eye drops containing lytic enzymes of Hemophilus, Pseudomonas, and/or Staphylococcus can be used to directly treat eye infections. Treatment with lytic enzymes are faster and more expedient than with antibiotics.

In yet another embodiment of the invention the phage associated lytic enzyme(s) is (are) put into a carrier which is placed in an inhaler to treat or prevent the spread of diseases localized in the mucus lining of the oral cavity and lungs. Specific lytic enzymes for tuberculosis have been isolated and can be used.

In another embodiment of the invention the lytic enzymes, shuffled lytic enzymes, and/or chimeric lytic enzymes, possibly with holin lytic enzymes, are administered in the form of a candy, chewing gum, lozenge, troche, tablet, a powder, an aerosol, a liquid, a liquid spray, or toothpaste for the prevention or treatment of bacterial infections associated with upper respiratory tract illnesses.

In another embodiment of the invention, species specific lytic enzymes can be used in the treatment of bacterial infections associated with topical or dermatological infections, administered in the form of a topical ointment or cream. In another embodiment of the invention, the lytic enzyme would be administered in an aqueous form. In yet another embodiment of the invention, lysostaphin, the enzyme which lyses Staphylococcus aureus, can be included in the therapeutic agent. In a further embodiment of the invention, conventional antibiotics may be included in the therapeutic agent with the lytic enzyme, and with or without the presence of lysostaphin. More than one lytic enzyme may also be included in the prophylactic or therapeutic agent.

### BRIEF DESCRIPTION OF THE DRAWINGS

FIG. 1 is an electron micrograph of group A streptococci treated with lysin showing the collapse of the cell wall and the cell contents pouring out;

FIG. 2 is a graph for the killing of S. pneumoniae (#DCC 1490) serotype 14 with PAL at various dilutions;

FIG. 3 is a graph showing the the decrease of bacterial titer within 30 seconds after addition of 100 U Pal phage enzyme;

FIG. 4 is a series of graphs showing the decrease of the Bacterial titer with 30 seconds after the addition of 100, 1,000, and 10,000 U Pal Lytic Enzyme; and

FIG. 5 is a series of graphs showing the decrease of bacterial titer within 30 seconds after addition of different amounts of U Pal.

### DETAILED DESCRIPTION OF THE INVENTION

The method for treating bacterial infections comprises treating the infection with a therapeutic agent comprising an effective amount of at least one lytic enzyme produced by a bacteria infected with a bacteriophage specific for the bacteria wherein at least one lytic enzyme is selected from the group consisting of shuffled lytic enzymes, chimeric lytic enzymes, and combinations thereof. The lytic enzyme is preferably in an environment having a pH which allows for activity of said lytic enzyme. A holin enzyme may be used in conjunction with the administration of the modified lytic enzyme. The holin enzyme may be in its “natural” state, may be shuffled holin enzymes or may be chimeric lytic enzymes.

Additionally, therapeutic compositions of this invention include one or more bacteria-associated phage enzymes, including isozymes, analogs, or variants thereof, in a natural or modified form. The modified form of the enzyme, for example, shuffled and/or chimeric enzymes, is produced enzymatically by chemical synthesis and/or DNA recombination technology.

The invention features the use of the chimeric and shuffled lytic and holin enzymes, as examples of bacteria-associated phage enzymes, in the therapeutic compositions and methods disclosed. These enzymes are used, for example, in the treatment or prevention of, for example, Streptococcal pygenes, Hemophilus influenza, Pseudomonas, Streptococcus pneumoniae, Streptococcus fasciae, Streptococcus group B, Listeria, Salmonella, E. coli, Campylobacter, Mycobacteria tuberculosis Staphylococcu, Helicobacter pylori or combinations thereof.

The lytic enzymes, shuffled lytic enzymes, chimeric lytic enzymes, as well as, or in conjunction with holin lytic enzymes, can be used for the treatment or prevention of Hemophilus influenza, Pseudomonas, Streptococcus pneumoniae, Streptococcus fasciae, Streptococcus group B, Listeria, Salmonella, E. coli, Campylobacter, and other bacteria, and any combination thereof. This lytic enzyme(s) may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme.

It should be understood that bacteriophage lytic enzyme are enzymes that specifically cleave bonds that are present in the peptidoglycan of bacterial cells. Since the bacterial cell wall peptiodglycan is highly conserved among all bacteria, there are only a few bonds to be cleaved to disrupt the cell wall. Enzymes that cleave these bonds are muramidases, glucosaminidases, endopeptidases, or N-acetyl-muramoyl L alanine amidases (hereinafter referred to as amidases). The majority of reported phage enzymes are either muramidases or amidases, and there have been no reports of bacteriophage glucosaminidases. Fischetti et al (1974) reported that the C1 streptococcal phage lysin enzyme was an amidase. Garcia et al (1987, 1990) reported that the Cp-1 lysin from a S pneumoniae phage was a muramidase. Caldentey and Bamford (1992) reported that a lytic enzyme from the phi 6 Pseudomonas phage was an endopeptidase, splitting the peptide bridge formed by meso-diaminopimilic acid and D-alanine. The E. coli T1 and T6 phage lytic enzymes are amidases as is the lytic enzyme from Listeria phage (ply) (Loessner et al, 1996).

There are a large number of phages which will attach to specific bacteria and produce enzymes which will lyse that particular bacteria. The following are a list of bacteriophages and bacteria for which they are specific:

Streptococci

Pseudomonas

Pneumococci

Salmonella

Staphylococci

Shigella

Haemophilus

Listeria

Mycobacteria

Vibrio

Corynebacteria

Bacillus

Spirochete

Myxococcus

Burkholderia

Brucella

Yersinia

Clostridium

Campylobacter

Neisseria

Actinomycetes

Agrobacterium

Alcaligenes

Clostridium

Coryneforms

Cyanobacteria

Enterobacteria

Lactobacillus

Lactoctococcus

Micrococcus

Pasteurella

Rhizobium

Xanthomonas

Bdellovibrio

mollicutes

Chlamydia

Spiroplasma

Caulobacter

Various phages which can be used to infect these bacteria and create the lytic enzyme include:

| BACTERIA | PHAGE(S) |
|---|---|
| Actinomycetes | A1-Dat, Bir, M1, MSP8, P-a-1, R1, R2, SV2, |
| | VP5, PhiC, φ31C, φUW21, φ115-A, φ150A, 119, |
| | SK1, 108/016 |
| Aeromonas | 29, 37, 43, 51, 59.1 |
| Altermonas | PM2 |
| Bacillus | AP50, φNS11, BLE, Ipy-1, MP15, mor1, PBP1, |
| | SPP1, Spbb, type F, alpha, φ105, 1A, II, Spy-2, |
| | SST, G, MP13, PBS1, SP3, SP8, SP10, SP15, |
| | SP50 |
| Bdellovibrio | MAC-1, MAC-1′, MAC-2, MAC-4, |
| | MAC-4′, MAC-5, MAC-7 |
| Caulobacter | φCb2, φCb4, φCb5, φCb8r, φCb9, φCB12r, |
| | φCb23r, φCP2, φCP18, φCr14, φCr28, PP7, |
| | φCb2, φCb4, φCb5, φCb8r, φCb9, φCB12r, |
| | φCb23r, φCP2, φCP18, φCr14, φCr28, PP7 |
| Chlamydia | Chp-1 |
| Clostridium | F1, HM7, HM3, CEB, |
| Coliform | AE2, dA, Ec9, fl, fd, HR, M13, ZG/2, ZJ/2 |
| Coryneforms | Arp, BL3, CONX, MT, Beta, A8010, A19 |
| Cyanobacteria | S-2L, S-4L, N1, AS-1, S-6(L) |
| Enterobacter | C-2, If1, If2, Ike, 12-2, PR64FS, SF, tf-1, PRD1, |
| | H-19J, B6, B7, C-1, C2, Jersey, ZG/3A, T5, ViII, |
| | b4, chi, Beccles, tu, PRR1, 7s, C-1, c2, fcan, |
| | folac, Ialpha, M, pilhalpha, R23, R34, ZG/1, |
| | ZIK/1, ZJ/1, ZL/3, ZS/3, alpha15, f2, fr, FC3-9, |
| | K19, Mu, 01, P2, ViI, φ92, 121, 16-19, 9266, |
| | C16, DdVI, PST, SMB, SMP2, a1, 3, 3T+, 9/0, |
| | 11F, 50, 66F, 5845, 8893, M11, QB, ST, TW18, |
| | VK, FI, ID2, fr, f2, |
| Listeria | H387, 2389, 2671, 2685, 4211 |
| Micrococcus | N1, N5 |
| Mycobacterium | Lacticola, Leo, R1-Myb, 13 |
| Pasteurella | C-2, 32, AU |
| Pseudomonas | Phi6, Pf1, Pf2, Pf3, D3, Kf1, M6, PS4, SD1, |
| | PB-1, PP8, PS17, nKZ, nW-14, n1, 12S, |
| Staphyloccous | 3A, B11-M15, 77, 107, 187, 2848A, Twort |
| Streptococcus | A25, A25 PE1, A25 VD13, A25 omega8, A25 |
| | 24 |
| Steptococcus A | |
| Vibrio | OXN-52P, VP-3, VP5, VP11, alpha3alpha, IV, kappa, |
| 06N- | 22-P, VP1, x29, II, nt-1, |
| Xanthomonas | Cf, Cf1t, Xf, Xf2, XP5 |

There are numerous other phages infecting these and other bacteria. The bacteriophages are normally grouped into family, genus and species, including Genus Chlamydiamicrovirus, Genus Bdellomicrovirus, Genus Spiromicrovirus, Genus Microvirus, Genus Microvirus, Genus Levivirus, Genus Allolevivirus, and other genuses.

The DNA coding of these phages and other phages may be altered to allow a recombinant enzyme to attack one cell wall at more than two locations, to allow the recombinant enzyme to cleave the cell wall of more than one species of bacteria, to allow the recombinant enzyme to attack other bacteria, or any combinations thereof. The type and number of alterations to a recombinant bacteriophage produced enzyme are incalculable.

For example, if there is a bacterial infection of the upper respiratory tract, the infection can be prophylactically or therapeutically treated with a composition comprising an effective amount of at least one lytic enzyme produced by a bacteria being infected with a bacteriophage specific for that bacteria, and a carrier for delivering the lytic enzyme to a mouth, throat, or nasal passage. The lytic enzyme is preferably a chimeric and/or shuffled lytic enzyme which may be used in conjunction with a holin enzyme or modified or unmodified phage associated lytic enzyme. It is also preferred that the lytic enzyme is in an environment having a pH which allows for activity of the lytic enzyme. If an individual has been exposed to someone with the upper respiratory disorder, the lytic enzyme will reside in the mucosal lining and prevent any colonization of the infecting bacteria.

Two examples of bacteria which infect the upper respiratory system are Streptococcus pneumoniae and Hemophilus influenzae. In recent years, there has been an increase in the number of people, particularly children and the elderly, that are infected or are carriers of penicillin resistant Streptococcus pneumoniae and Hemophilus. While these bacteria are normally harmless residents of the host, they are opportunistic organisms that are able to cause infections when the resistance of the host has been compromised. By eliminating or reducing the number of these organisms in the upper respiratory tract, there will be a commensurate reduction in the number of infections by these bacteria.

Infection of the Hemophilus bacteria by Bacteriophage HP1 (a member of the P2-like phage family with strong similarities to coliphages P2 and 186, and some similarity to the retronphage Ec67) produces a lytic enzyme capable of lysing the bacteria. The lytic enzyme for Streptococcus pneumoniae, previously identified as a -acetyl-muramoyl-L-alanine amidase, is produced by the infecting Streptococcus pneumoniae with the Pal bacteriophage. The therapeutic agent can contain either or both of the lytic enzymes produced by these two bacteria, and may contain other lytic enzymes for other bacteria. The composition which may be used for the prophylactic and therapeutic treatment of a strep infection includes the lysin enzyme and a means of application, (such as a carrier system or an oral delivery mode), to the mucosal lining of the oral and nasal cavity, such that the enzyme is put in the carrier system or oral delivery mode to reach the mucosal lining. Another infection which can be treated prophylactically is Streptococcus group A, which can produce what is commonly known as “strep” throat. When group C Streptococci are infected with a C1 bacteriophage, a lysin enzyme is produced specific for the lysing of Streptococcus group A.

While “nonrecombinant” or “unmodified” phage associated lytic enzymes may be used for treatment of the Streptococcus, it is preferred that a shuffled or chimeric lytic enzyme be used, possibly with a holin enzyme.

Prior to, or at the time the lysin enzyme is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

Means of application include, but are not limited to direct, indirect, carrier and special means or any combination of means. Direct application of the enzyme may be by nasal sprays, nasal drops, nasal ointments, nasal washes, nasal injections, nasal packings, bronchial sprays and inhalers, or indirectly through use of throat lozenges, or through use of mouthwashes or gargles, or through the use of ointments applied to the nasal nares, the bridge of the nose, or the face or any combination of these and similar methods of application. The forms in which the lysin enzyme may be administered include but are not limited to lozenges, troches, candies, injectants, chewing gums, tablets, powders, sprays, liquids, ointments, and aerosols.

The lozenge, tablet, or gum into which the enzymes are added may contain sugar, corn syrup, a variety of dyes, non-sugar sweeteners, flavorings, any binders, or combinations thereof. Similarly, any gum based products may contain acacia, carnauba wax, citric acid, corn starch, food colorings, flavorings, non-sugar sweeteners, gelatin, glucose, glycerin, gum base, shellac, sodium saccharin, sugar, water, white wax, cellulose, other binders, and combinations thereof.

Lozenges may further contain sucrose, corn starch, acacia, gum tragacanth, anethole, linseed, oleoresin, mineral oil, and cellulose, other binders, and combinations thereof. In another embodiment of the invention, sugar substitutes are used in place of dextrose, sucrose, or other sugars.

The enzyme may also be placed in a nasal spray, wherein the nasal spray is the carrier. The nasal spray can be a long acting or timed release spray, and can be manufactured by means well known in the art. An inhalant may also be used, so that the phage enzyme may reach further down into the bronchial tract, including into the lungs.

Any of the carriers for the lytic enzymes may be manufactured by conventional means. However, it is preferred that any mouthwash or similar type products not contain alcohol to prevent denaturing of the enzyme. Similarly, when the lytic enzymes are being placed in a cough drop, gum, candy or lozenge during the manufacturing process, such placement should be made prior to the hardening of the lozenge or candy but after the cough drop or candy has cooled somewhat, to avoid heat denaturation of the enzyme.

The enzyme may be added to these substances in a liquid form or in a lyophilized state, whereupon it will be solubilized when it meets body fluids such as saliva. The enzyme may also be in a micelle or liposome.

The effective dosage rates or amounts of the lytic enzyme(s) to treat the infection will depend in part on whether the lytic will be used therapeutically or prophylactically, the duration of exposure of the recipient to the infectious bacteria, the size and weight of the individual, etc. The duration for use of the composition containing the enzyme also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of enzyme(s) believed to provide for an effective amount or dosage of enzyme may be in the range of about 100 units/ml to about 100,000 units/ml of fluid in the wet or damp environment of the nasal and oral passages, and possibly in the range of about 100 units/ml to about 10,000 units/ml. More specifically, time exposure to the active enzyme units may influence the desired concentration of active enzyme units per ml. It should be noted that carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per ml and the duration of time of exposure depends on the nature of infection, whether treatment is to be prophylactic or therapeutic, and other variables.

While this treatment may be used in any mammalian species, the preferred use of this product is for a human.

This composition and method may also be used for the treatment of Streptococcus A infections of the respiratory tract. When using this composition for a Streptococcus A infection, the chimeric and/or shuffled lytic enzymes should be used for the prophylactic prevention of Streptococcus infections. Similarly, in another embodiment of the invention, this method may be used for the therapeutic and, preferably, the prophylactic treatment of tuberculosis. In a preferred embodiment of the invention, the phage associated lysing enzyme for Mycobacteria tuberculosis is placed in a carrier in an inhaler. The carrier may be sterile water or a water base, or any other carrier used in an inhaler for dispersing drugs into the bronchial tract. The phage associated chimeric and/or shuffled lytic enzyme specific for tuberculosis is subject to the same conditions as the phage associated lytic enzyme for other lytic enzymes. Specifically, prior to, or at the time the enzyme is put in the carrier system or oral delivery mode, it is preferred that the enzyme be in a stabilizing buffer environment for maintaining a pH range between about 4.0 and about 9.0.

The stabilizing buffer should allow for the optimum activity of the lytic enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

For the prophylactic and therapeutic treatment of tuberculosis, the phage associated chimeric and/or shuffled lytic enzymes associated with tuberculosis may also be applied by direct, indirect, carriers and special means or any combination of means. Direct application of the lytic enzyme may be by nasal sprays, nasal drops, nasal ointments, nasal washes, nasal injections, nasal packings, bronchial sprays and inhalers, or indirectly through use of throat lozenges, or through use of mouthwashes or gargles, or through the use of ointments applied to the nasal nares, the bridge of the nose, or the face or any combination of these and similar methods of application. The forms in which the lytic enzyme may be administered include but are not limited to lozenges, troches, candies, injectants, chewing gums, tablets, powders, sprays, liquids, ointments, and aerosols. For the therapeutic treatment of tuberculosis, the bronchial sprays and aerosols are most beneficial, as these carriers, or means of distributing the composition, allow the lytic enzyme to reach the bronchial tubes and the lungs. An appropriate transport carrier may be attached to the enzyme to transport the enzyme across the cell membrane to the site of the bacteria. The chimeric and/or shuffled lytic enzymes may be used in combination with other chimeric and shuffled lytic enzymes, holin enzymes, other lytic enzymes, and other phage associated lytic enzymes which have not been modified or which are not “recombinant.”

Another use of a lytic enzyme is for the treatment of bacterial infections of the digestive tract. The method for treating a bacterial infection of the digestive tract comprises treating the bacterial infection with composition comprising an effective amount of at least one lytic enzyme produced by a bacteria infected with a bacteriophage specific for the bacteria, and a carrier for delivering said lytic enzyme to the digestive tract. In a preferred embodiment of the invention, the bacterial infections being treated are selected from the group consisting of H., pyogenes, Listeria, Salmonella, E. coli, and Campylobacter. However, this method and composition will effectively treat other bacteria, when the appropriate lytic enzyme is used. The lytic enzymes used in the digestive tract may be either supplemented by chimeric and/or shuffled lytic enzymes, or may be themselves chimeric and/or shuffled lytic enzymes. Similarly, a holin enzyme may be included, which may also be a chimeric and/or shuffled lytic enzyme.

In a preferred embodiment of the invention, the carrier is selected from the group consisting of suppository enemas, syrups, or enteric coated pills. These proposed carriers can be made by conventional methods. However, the only difference in their manufacture is that the enzyme being placed in the carrier must not be allowed to denature. The enzyme should be incorporated into a carrier which does not contain alcohol, and which has been cooled to a temperature that will not cause the denaturing of the enzyme. The enzyme may be incorporated in a lyophilized state, or may be incorporated in a liposome before being placed in the suppository, syrup or enteric coated pill. The enzyme placed in the composition or carrier should be in an environment having a pH which allows for activity of the lytic enzyme. To this end, the pH of the composition is preferably kept in a range of between about 2 and about 11, more preferably in a range of between about between about 4.0 and about 9.0, and even more preferably at a pH range of between about 5.5 and about 7.5. As described above with the other lytic enzyme, the pH can be moderated by the use of a buffer. The buffer may contain a reducing agent, and more specifically dithiothreitol. The buffer may also be a metal chelating reagent, such as ethylenediaminetetracetic disodium salt or the buffer may contain a citrate-phosphate buffer. As with all compositions described in this patent, the composition may, further include a bactericidal or bacteriostatic agent as a preservative.

The lytic enzyme(s) preferably are present in a concentration of about 100 to about 500,000 active enzyme units per milliliter of fluid in the wet environment of the gastrointestinal tract, preferably about 100 to about 100,000 active enzyme units per milliliter of fluid, and preferably present in a concentration of about 100 to about 10,000 active enzyme units per milliliter of fluid in the wet environment of the gastrointestinal tract.

The suppository is known in the art, and is made of glycerin, fatty acids, and similar type substances that dissolve at body temperature. As the suppository dissolves, the phage associated lytic enzyme will be released.

Another composition and use of the lytic enzyme is for the therapeutic or prophylactic treatment of bacterial infections of burns and wounds of the skin. The composition comprises an effective amount of at least one lytic enzyme produced by a bacteria infected with a bacteriophage specific for the bacteria and a carrier for delivering at least one lytic enzyme to the wounded skin. The lytic enzyme(s) used for the topical treatment of burns may be either supplemented by chimeric and/or shuffled lytic enzymes, or may themselves be chimeric and/or shuffled lytic enzymes. Similarly, a holin enzyme may be included, which may also be a chimeric and/or shuffled lytic enzyme. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes but is not limited to a smear, spray, a time-release patch, a liquid absorbed wipe, and combinations thereof. The lytic enzyme may be applied to a bandage either directly or in one of the other carriers. The bandages may be sold damp or dry, wherein the enzyme is in a lyophilized form on the bandage. This method of application is most effective for the treatment of burns.

The carriers of the compositions of the present invention may comprise semisolid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament.

Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises !“CARBOPOL.RTM.” (B. F. Goodrich, Cleveland, Ohio), “HYPAN.RTM.” (Kingston Technologies, Dayton, N.J.), “NATROSOL.RTM.” (Aqualon, Wilmington, Del.), “KLUCEL.RTM.” (Aqualon, Wilmington, Del.), or “STABILEZE.RTM.” (ISP Technologies, Wayne, N.J.). Preferably, the gelling agent comprises between about 0.2% to about 4% by weight of the composition. More particularly, the preferred compositional weight percent range for “CARBOPOL.RTM.” is between about 0.5% to about 2%, while the preferred weight percent range for “NATROSOL.RTM.” and “KLUCEL.RTM.” is between about 0.5% to about 4%. The preferred compositional weight percent range for both “HYPAN.RTM.” and “STABILEZE.RTM.” is between about 0.5% to about 4%. CARBOPOL.RTM.” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL.RTM.” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

Preservatives may also be used in this invention and preferably comprise about 0.05% to 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Titanium dioxide may be used as a sunscreen to serve as prophylaxis against photosensitization. Alternative sun screens include methyl cinnamate. Moreover, BHA may be used as an antioxidant, as well as to protect ethoxydiglycol and/or dapsone from discoloration due to oxidation. An alternate antioxidant is BHT.

Pharmaceuticals for use in all embodiments of the invention include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%.Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide are recommended at concentrations of about 0.01% to 1.0% by weight. Preferred concentrations for corticosteroids such as hydrocortisone or methylprednisolone acetate are from about 0.2% to about 5.0% by weight.

Destructive therapy agents such as salicylic acid or lactic acid may also be used. A concentration of about 2% to about 40% by weight is preferred. Cantharidin is preferably utilized in a concentration of about 5% to about 30% by weight. Typical antifungals that may be used in this invention and their preferred weight concentrations include: oxiconazole nitrate (0.1% to 5.0%), ciclopirox olamine (0.1% to 5.0%), ketoconazole (0.1% to 5.0%), miconazole nitrate (0.1% to 5.0%), and butoconazole nitrate (0.1% to 5.0%). For the topical treatment of seborrheic dermatitis, hirsutism, acne, and alopecia, the active pharmaceutical may include an antiandrogen such as flutamide or finasteride in preferred weight percentages of about 0.5% to 10%.

Typically, treatments using a combination of drugs include antibiotics in combination with local anesthetics such as polymycin B sulfate and neomycin sulfate in combination with tetracaine for topical antibiotic gels to provide prophylaxis against infection and relief of pain. Another example is the use of minoxidil in combination with a corticosteroid such as betamethasone diproprionate for the treatment of alopecia ereata. The combination of an anti-inflammatory such as cortisone with an antifungal such as ketoconazole for the treatment of tinea infections is also an example.

In one embodiment, the invention comprises a dermatological composition having about 0.5% to 10% carbomer and about 0.5% to 10% of a pharmaceutical that exists in both a dissolved state and a micro particulate state. The dissolved pharmaceutical has the capacity to cross the stratum corneum, whereas the micro particulate pharmaceutical does not. Addition of an amine base, potassium, hydroxide solution, or sodium hydroxide solution completes the formation of the gel. More particularly, the pharmaceutical may include dapsone, an antimicrobial agent having anti-inflammatory properties. A preferred ratio of micro particulate to dissolved dapsone is five or less.

In another embodiment, the invention comprises about 1% carbomer, about 80-90% water, about 10% ethoxydiglycol, about 0.2% methylparaben, about 0.3% to 3.0% dapsone including both micro particulate dapsone and dissolved dapsone, and about 2% caustic material. More particularly, the carbomer may include “CARBOPOL.RTM. 980” and the caustic material may include sodium hydroxide solution.

In a preferred embodiment, the composition comprises dapsone and ethoxydiglycol, which allows for an optimized ratio of micro particulate drug to dissolved drug. This ratio determines the amount of drug delivered, compared to the amount of drug retained in or above the stratum corneum to function in the supracorneum domain. The system of dapsone and ethoxydiglycol may include purified water combined with “CARBOPOL.RTM.” gelling polymer, methylparaben, propylparaben, titanium dioxide, BHA, and a caustic material to neutralize the “CARBOPOL.RTM.”

Any of the carriers for the lytic enzyme may be manufactured by conventional means. However, if alcohol is used in the carrier, the enzyme should be in a micelle, liposome, or a “reverse” liposome, to prevent denaturing of the enzyme. Similarly, when the lytic enzyme is being placed in the carrier, and the carrier is, or has been heated, such placement should be made after the carrier has cooled somewhat, to avoid heat denaturation of the enzyme. In a preferred embodiment of the invention, the carrier is sterile.

The enzyme may be added to these substances in a liquid form or in a lyophilized state, whereupon it will be solubilized when it meets a liquid body.

The effective dosage rates or amounts of the lytic enzyme to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml and the duration of time of exposure depends on the nature of infection, and the amount of contact the carrier allows the lytic enzyme(s) to have. It is to be remembered that the enzyme works best when in a fluid environment. Hence, effectiveness of the enzyme(s) is in part related to the amount of moisture trapped by the carrier. In another preferred embodiment, a mild surfactant in an amount effective to potentiate the therapeutic effect of the lytic enzyme. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate.

In order to accelerate treatment of the infection, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate, chelating agents, streptomycin, erythromycin, chloramphenicol, numerous other antibiotics, and any combinations thereof in amounts which are effective to synergistically enhance the therapeutic effect of the lytic enzyme. It should be noted that virtually any antibiotic may be used as complementary agents for or with any use of the recombinant lytic enzymes.

Additionally, the therapeutic agent may further comprise the enzyme lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the lysin enzyme, can be used in the presence or absence of the listed antibiotics. There is a degree of added importance in using both lysostaphin and the lysin enzyme in the same therapeutic agent. Frequently, when a body has a bacterial infection, the infection by one genus of bacteria weakens the body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other gram positive bacterial strains will not be killed by standard antibiotics. Consequently, the use of the lysin and lysostaphin, possibly in combination with antibiotics, can serve as the most rapid and effective treatment of bacterial infections. In yet another preferred embodiment, the invention may include mutanolysin, and lysozyme.

In preferred embodiments of the invention, the chimeric and/or shuffled lytic enzymes for Pseudomonas, Staphylococcus, and Streptococcus, jointly or individually, may be incorporated into the carrier, or into a bandage to be used on burn patients, or in a solution or cream carrier. These enzymes may be used in combination with holin and other lytic enzymes.

Yet another use of lytic enzymes is for the prophylactic or therapeutic treatment of vaginal infections. This treatment comprises treating the vaginal infection with an effective amount of at least one lytic enzyme produced by a bacteria being infected with a bacteriophage specific for that bacteria, wherein that lytic enzyme is incorporated in a carrier to be placed in a vagina. The lytic enzyme(s) used to treat bacterial infections of the vagina may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme. The preferred carrier is a tampon, or vaginal douche. A pad may also be used as a carrier, although it is not as effective. While any number of bacteria could be treated using this composition and method, it is believed that the most optimum use of this treatment composition and method would be for the treatment of an E. coli and Streptococcus B infection. Vaginal infections caused by Group B Streptococcus can cause neonatal meningitis resulting in brain damage and premature death. Lytic enzymes incorporated into tampon specific for group B Strep would eliminate the group B organisms without disturbing normal flora so that woman would not be overcome by yeast infection post antibiotic therapy. The use of the lytic enzymes in the vagina would best provide a prophylactic effect, although therapeutic use would also be advisable.

To produce a pad or tampon containing the enzyme, the lytic enzymes can be applied in a solution to the tampon, and allowed to dry. The lytic enzyme may be incorporated into the pad or tampon by any other means known in the art, including lyophilization, spraying, etc. The tampons and pads may also be kept slightly moist, and in a sealed wrapper until ready for use. In that case, bactericide and bacteriostatic compounds and inhibitors should be present in the tampons and pads. The method to be used for incorporating the lytic enzyme into the tampon or pad can be one of the methods known in the art for incorporating a pharmaceutical product. In another embodiment of the invention, the lytic enzyme is incorporated into a vaginal suppository. The vaginal suppository into which the lytic enzyme is being incorporated may be a standard vaginal suppository, comprised of glyceride, alginate, starch, other standard binders and any combinations thereof.

When using a tampon as the carrier, it is best to insert the tampon in the vagina and leave it in for up to 12 hours to distribute the enzyme vaginally.

As with other lytic enzymes, it is preferable that the pH be kept in a range of about 4.0 and about 9.0 even more preferably at a pH range of between about 5.5 and about 7.5. As described above with the other lytic enzyme, the pH can be moderated by the use of a buffer. The buffer may contain a reducing agent, and more specifically dithiothreitol. The buffer may also contain a metal chelating reagent, such as ethylenediaminetetracetic disodium salt or the buffer may be a citrate-phosphate buffer. As with all compositions described in this patent, the composition may, further include a bactericidal or bacteriostatic agent as a preservative.

The lytic enzyme(s) are preferably present in a concentration of about 100 to about 500,000 active enzyme units per milliliter of fluid in the wet environment of the vaginal tract, preferably about 100 to about 100,000 active enzyme units per milliliter of fluid, and preferably present in a concentration of about 100 to about 10,000 active enzyme units per milliliter of fluid in the wet environment of the vaginal tract.

Another use of the invention is for the prophylactic and therapeutic treatment of eye infections. The method of treatment comprises administering eye drops which comprise an effective amount of at least one lytic enzyme produced by the bacteria being infected with a bacteriophage specific for the bacteria and a carrier capable of being safely applied to an eye, with the carrier containing the lytic enzyme. In a preferred embodiment of the invention, the bacteria being treated is Hemophilus or Staphylococcus The eye drops are in the form of an isotonic solution. The pH of the solution should be adjusted so that there is no irritation of the eye, which in turn would lead to possibly infection by other organisms, and possibly to damage to the eye. While the pH range should be in the same range as for other lytic enzymes, the most optimal pH will be in the range of from 6.0 to 7.5. Similarly, buffers of the sort described above for the other lytic enzymes should also be used. Other antibiotics which are suitable for use in eye drops may be added to the composition containing the lytic enzymes. Bactericides and bacteriostatic compounds may also be added. As stated above, this lytic enzyme may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme.

It is to be remembered that all of the enzymes can be used for prophylactic and therapeutic treatments of the bacteria for which the enzymes are specific.

Additionally, a carrier may have more than one lytic enzyme. For instance, a throat lozenge may comprise just a lysin enzyme (which lyses the Streptococcus A strain causing “strep” throat) or it may also include the lytic enzymes for Hemophilus. Similarly, the carrier for treating burns and wounds, or infections of the skin, may contain just one lytic enzyme, or a combination of lytic enzymes, for the treatment of Pseudomonas, Streptococcus, Staphylococcus, or any other of a number of bacteria. The carrier may include any combination of lytic enzymes, shuffled lytic enzymes, chimeric lytic enzymes, and holin enzymes.

Lytic enzymes can also be used to fight dental caries. See, for example, a lytic enzyme specific for Streptococcus mutans may be incorporated in a toothpaste or oral wash. Similarly, this lytic enzyme may also be incorporated into a chewing gum or lozenge. Any other carrier can be used that allows for the exposure of the mouth, gums, and teeth to the lytic enzyme.

The lytic enzyme may also be incorporated in a lyophilized or dried form in tooth powder. If the lytic enzyme is to be used in an oral wash, it is preferred that the oral wash not contain any alcohol, so as to not denature the enzyme. The enzyme can also be in a liposome when mixed in with the toothpaste or oral wash. The concentrations of the enzyme units per ml of toothpaste or mouth wash can be in the range of from about 100 units/ml to about 500,000 units/ml of composition, preferably in the range of about 1000 units/ml to about 100,000 units/ml, and most preferably from about 10,000 to 100,000 units/ml. The pH of the toothpaste or oral wash should be in a range that allows for the optimum performance of the enzyme, while not causing any discomfort to the user of the toothpaste or oral wash. Again, as with the other uses of lytic enzymes, the lytic enzyme use to treat dental caries may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme.

The lytic enzymes may also be administered parenterally. The lytic enzyme, holin lytic enzyme, chimeric enzyme, shuffled enzyme, and combinations thereof may be administered parenterally using an effective amount of a therapeutic agent, the therapeutic agent comprising at least one lytic enzyme produced by a bacteria infected with a bacteriophage specific for said bacteria selected from the group consisting of holin lytic enzymes, chimeric lytic enzymes, shuffled lytic enzymes, and combinations thereof, and a carrier for delivering the lytic enzyme to the site of the infection.

The composition may be used for the therapeutic treatment of Pseudomonas, Clostridium, Staphylococcus infections, among others.

A number of different bacteria may be treated. Among the bacteria which most often infect deep tissues, and, more specifically connective tissues, are Group A Streptococcus, Staphylococcus, Pseudomonas, and Clostridium. More than one lytic enzyme may be introduced into the infected body at a time.

A number of different methods may be used to introduce the lytic enzyme(s). These methods include introducing the lytic enzyme intravenously, intramuscularly, subcutaneously, and subdermally.

In one preferred embodiment of the invention, a deep tissue infection may be treated by injecting into the infected tissue of the patient a therapeutic agent comprising the appropriate lytic enzyme(s) (holin lytic enzyme, chimeric lytic enzyme and/or shuffled lytic enzyme) and a carrier for the enzyme. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein.

In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are provided sterile and pyrogen free.

The carrier suitably contains minor amounts of additives such as substances that enhance isotonicity and chemical stability. Such materials are nontoxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% and more, but preferably about 20%.

DMSO, an aprotic solvent with a remarkable ability to enhance penetration of many locally applied drugs, may be diluted in sterile water for injection, or sodium chloride injection, or other pharmaceutically acceptable aqueous injection fluid, and used in concentrations of 0.1 to 100% (v/v).

The carrier vehicle may also include Ringer's solution, a buffered solution, and dextrose solution, particularly when an intravenous solution is prepared.

Prior to, or at the time the lytic enzyme is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1. This is pH range is most suitable for the lysin enzyme for Streptococcus.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer. The buffers found in the carrier can serve to stabilize the environment for the lytic enzymes.

The effective dosage rates or amounts of the chimeric and/or shuffled lytic enzymes to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml and the duration of time of exposure depends on the nature of infection, and the amount of contact the carrier allows the lytic enzyme to have. It is to be remembered that the enzyme works best when in a fluid environment. Hence, effectiveness of the enzyme is in part related to the amount of moisture trapped by the carrier. For the treatment of septicemia, there should be a continuous intravenous flow of therapeutic agent into the blood stream. The concentration of lytic enzymes for the treatment of septicemia is dependent upon the seriousness of the infection.

In order to accelerate treatment of the infection, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate, chelating agents and any combinations thereof in amounts which are effective to synergistically enhance the therapeutic effect of the lytic enzyme. As previously noted, virtually any antibiotic may be used with the the various lytic enzymes, which include the shuffled and/or chimeric lytic enzymes, the holin enzymes, etc.

Additionally, the therapeutic agent may further comprise the enzyme lysostaphin for the treatment of any Staphylococcus aureus bacteria. In yet another preferred embodiment, the invention may include mutanolysin, and lysozyme

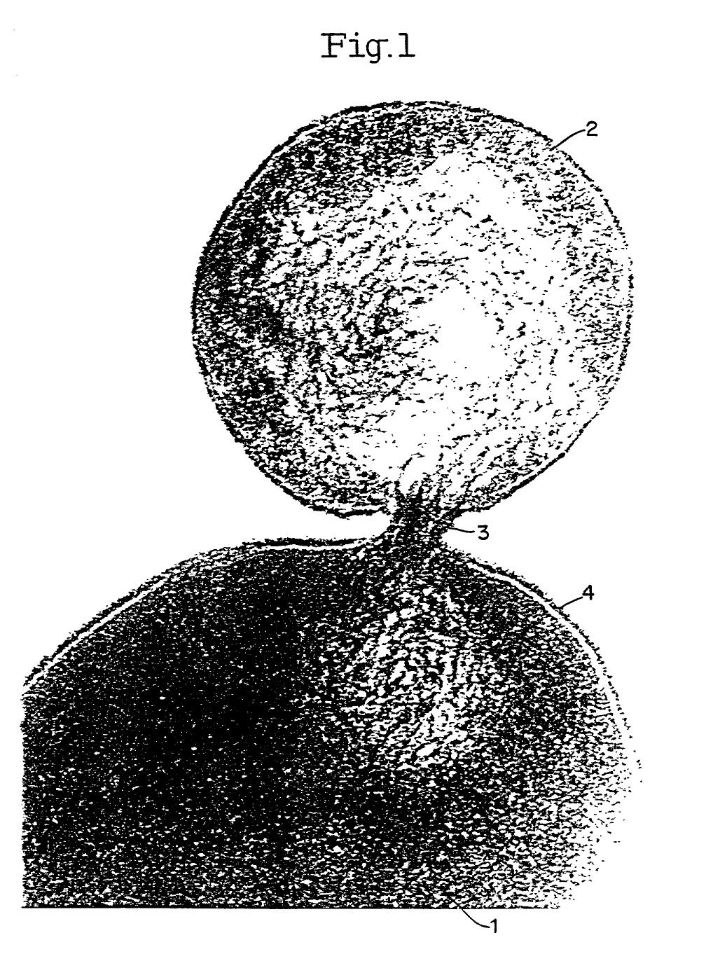

The use of lytic enzymes, including but not limited to holin lytic enzymes, chimeric lytic enzymes, shuffled lytic enzymes, and combinations thereof, rapidly lyse the bacterial cell. The thin section electron micrograph of FIG. 1 shows the results of a group A streptococci 1 treated for 15 seconds with lysin. The micrograph (25,000× magnification) shows the cell contents 2 pouring out through a hole 3 created in the cell wall 4 by the lysin enzyme.

As noted above, the use of the holin lytic enzyme, the chimeric lytic enzyme, and/or the shuffled lytic enzyme, may be accompanied by the use of a “natural” lytic enzyme, which has not been modified by the methods cited in U.S. Pat. No. 6,132,970, or by similar state of the art methods. The phage associated lytic enzyme may be prepared as shown in the following example:

### EXAMPLE 1

Harvesting Phage Associated Lytic Enzyme

Group C streptococcal strain 26RP66 (ATCC #21597) or any other group C streptococcal strain is grown in Todd Hewitt medium at 37.degree. C. to an OD of 0.23 at 650 nm in an 18 mm tube. Group C bacteriophage (Cl) (ATCC #21597-Bi) at a titer of 5.times.10.sup.6 is added at a ratio of 1 part phage to 4 parts cells. The mixture is allowed to remain at 37.degree. C. for 18 min at which time the infected cells are poured over ice cubes to reduce the temperature of the solution to below 15.degree. C. The infected cells are then harvested in a refrigerated centrifuge and suspended in 1/300th of the original volume in 0.1M phosphate buffer, pH 6.1 containing 5.times.10.sup.-3 M dithiothreitol and 10 ug of DNAase. The cells will lyse releasing phage and the lysin enzyme. After centrifugation at 100,000.times. g for 5 hrs to remove most of the cell debris and phage, the enzyme solution is aliquoted and tested for its ability to lyse Group A Streptococci.

The number of units/ml in a lot of enzyme is determined to be the reciprocal of the highest dilution of enzyme required to reduce the OD650 of a suspension of group A streptococci at an OD of 0.3 to 0.15 in 15 minutes. In a typical preparation of enzyme 4.times.10.sup.5 to 4.times.10.sup.6 units are produced in a single 12 liter batch.

Use of the enzyme in an immunodiagnostic assay requires a minimum number of units of lysin enzyme per test depending on the incubation times required. The enzyme is diluted in a stabilizing buffer maintaining the appropriate conditions for stability and maximum enzymatic activity, inhibiting nonspecific reactions, and in some configurations contains specific antibodies to the Group A carbohydrate. The preferred embodiment is to use a lyophilized reagent which can be reconstituted with water. The stabilizing buffer can comprise a reducing reagent, which can be dithiothreitol in a concentration from 0.001M to 1.0M, preferably 0.005M. The stabilizing buffer can comprise an immunoglobulin or immunoglobulin fragments in a concentration of 0.001 percent to 10 percent, preferably 0.1 percent. The stabilizing buffer can comprise a citrate-phosphate buffer in a concentration from 0.001M to 1.0M, preferably 0.05M. The stabilizing buffer can have a pH value in the range from 5.0 to 9.0. The stabilizing buffer can comprise a bactericidal or bacteriostatic reagent as a preservative. Such preservative can be sodium azide in a concentration from 0.001 percent to 0.1 percent, preferably 0.02 percent.

The preparation of phage stocks for lysin production is the same procedure described above for the infection of group C streptococcus by phage in the preparation of the lysin enzyme. However, instead of pouring the infected cells over ice, the incubation at 37.degree. C. is continued for a total of 1 hour to allow lysis and release of the phage and the enzyme in the total volume. In order for the phage to be used for subsequent lysin production the residual enzyme must be inactivated or removed to prevent lysis from without of the group C cells rather than phage infection.

The use of chimeric or shuffled enzymes shows a great improvement as to the properties of the enzyme, as illustrated by the following examples:

### EXAMPLE 2

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. Jul. 1, 1998 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 3

Isolation of the Pal Lytic Enzyme:

Recombinant E.coli DH5 (pMSP11) containing the pal lytic enzyme gene were grown overnight, induced with lactose, pelleted, resupended in phosphate buffer, broken by sonication. After centrifugation, the Pal enzyme in the supernatant was purified in a single step using a DEAE-cellulose column and elution with choline. Protein content was analyzed with the Bradford method. Using this method, a single protein band was identified by SDS-PAGE.

### EXAMPLE 4

Killing Assay:

S. pneumoniae of various serotypes and 8 different viridans streptococi were grown overnight and for most assays diluted and re-grown for 6h to log phase of growth, pelleted and resupended in 0.9% saline to an OD@620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units.

One unit (U) of Pal was defined as the highest dilution at which Pal decreased the OD of a pneumococcal strain by half in 15 minutes.

### EXAMPLE 5

Production of Chimeric Lytic Enzymes

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. Jul. 1, 1998 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 6

Isolation of the Pal Lytic Enzyme

Recombinant E.coli DH5 (pMSP11) containing the pal lytic enzyme gene were grown overnight, induced with lactose, pelleted, resupended in phosphate buffer, broken by sonication. After centrifugation, the Pal enzyme in the supernatant was purified in a single step using a DEAE-cellulose column and elution with choline. Protein content was analyzed with the Bradford method. Using this method, a single protein band was identified by SDS-PAGE.

### EXAMPLE 7

Killing Assay

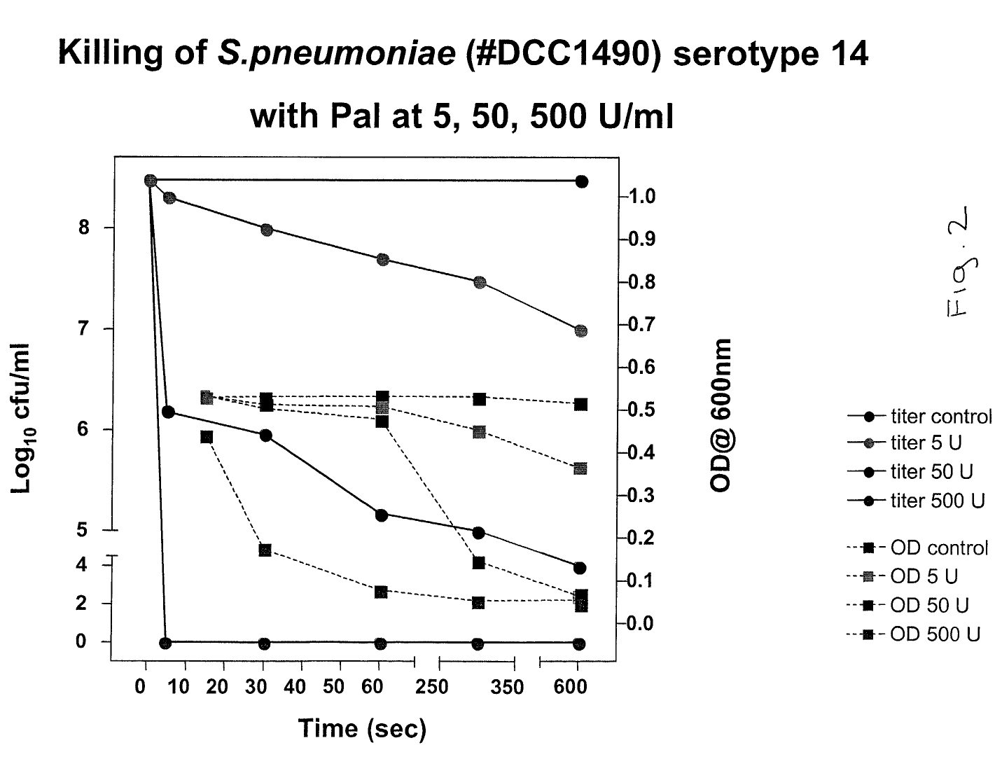

S. pneumoniae of various serotypes and 8 different viridans streptococi were grown overnight and for most assays diluted and re-grown for 6 h to log phase of growth, pelleted and resupended in 0.9% saline to an OD@620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units. One unit (U) of Pal was defined as the highest dilution at which Pal decreased the OD of a pneumococcal strain by half in 15 minutes. The results, (see FIG. 2) show that the viability of Pneumococci dropped more than 8 logs in five seconds after adding the Pal enzyme.

### EXAMPLE 8

Susceptability of Oral Streptoccocci to Pal Enzyme

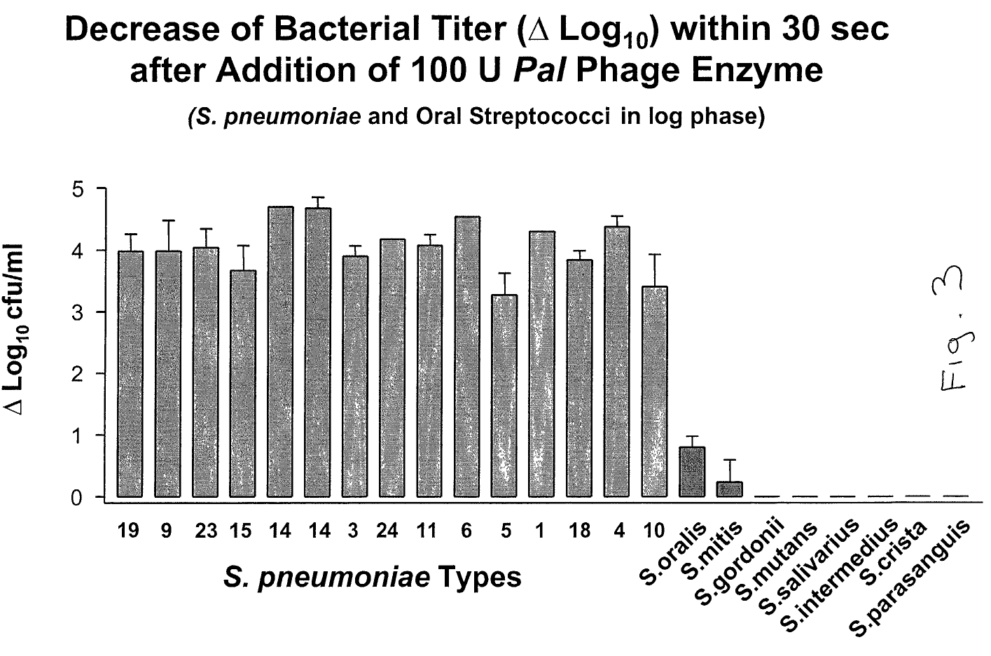

Various serotypes of oral streptoccoci were tested against bacteria-associated lytic enzymes, in particular, the Pal enzyme. A variety of S. pneumoniae type bacteria was also included in the test. Pal enzyme were used at a concentration of 100 U of the purified enzyme. As can be seen in FIG. 3 all S. pneumoniae serotypes are killed (˜4 logs) within the 30 seconds of exposure. Of the oral streptococci tested, only S. oralis and S. mitis show low sensitivity to the Pal enzyme.

### EXAMPLE 9

Susceptability of Stationary Phase Bacteria to Lytic Enzyme

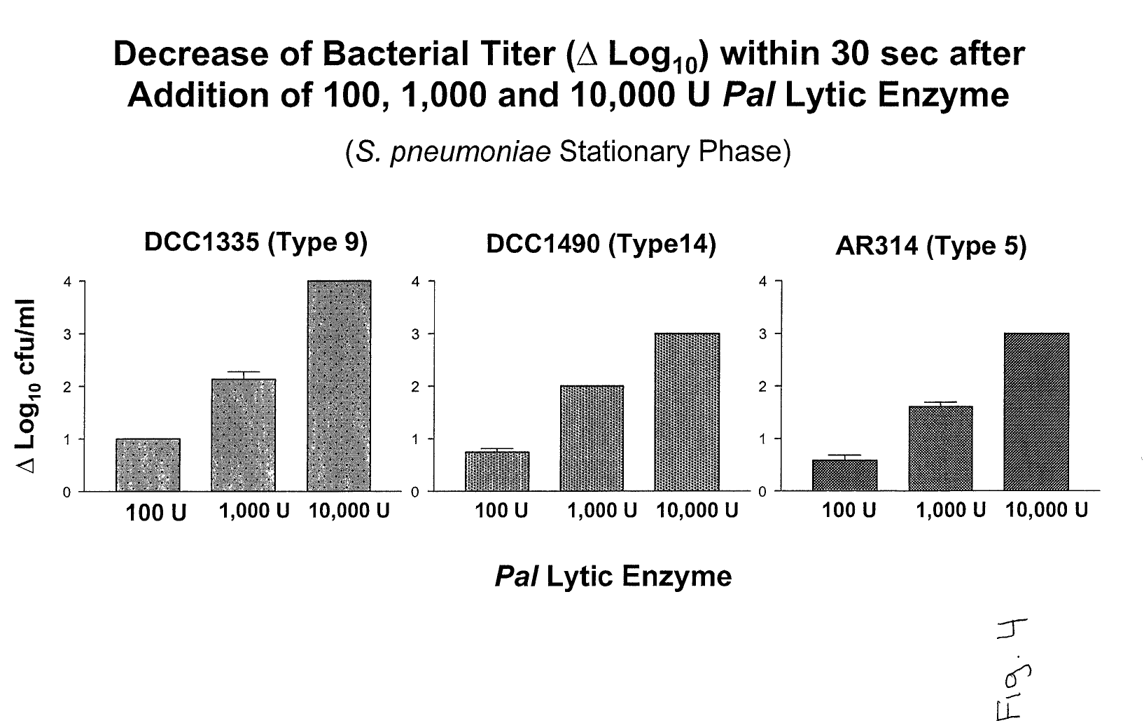

In order to confirm that activity of lytic enzymes are independent of the bacterial grwoth, several serotypes of serotypes of S.pneumoniae at stationary phase of growth were tested against lytic enzymes. In particular, 3 strains of Pal lytic enzyme were used against 3 sereotypes of S. pneumoniae. The results show that that all bacterial strains tested against Pal enzyme were killed in 30 seconds (see FIG. 4). An approximately 2-log drop in viability of the bacteria occurred with 1,000 U of enzyme, as opposed to about 3-4 log drop in the viability with 10,000 units.

### EXAMPLE 10

Effect of Pal Lytic Enzyme on Log-Phase and Stationary Phase Oral Streptococci.

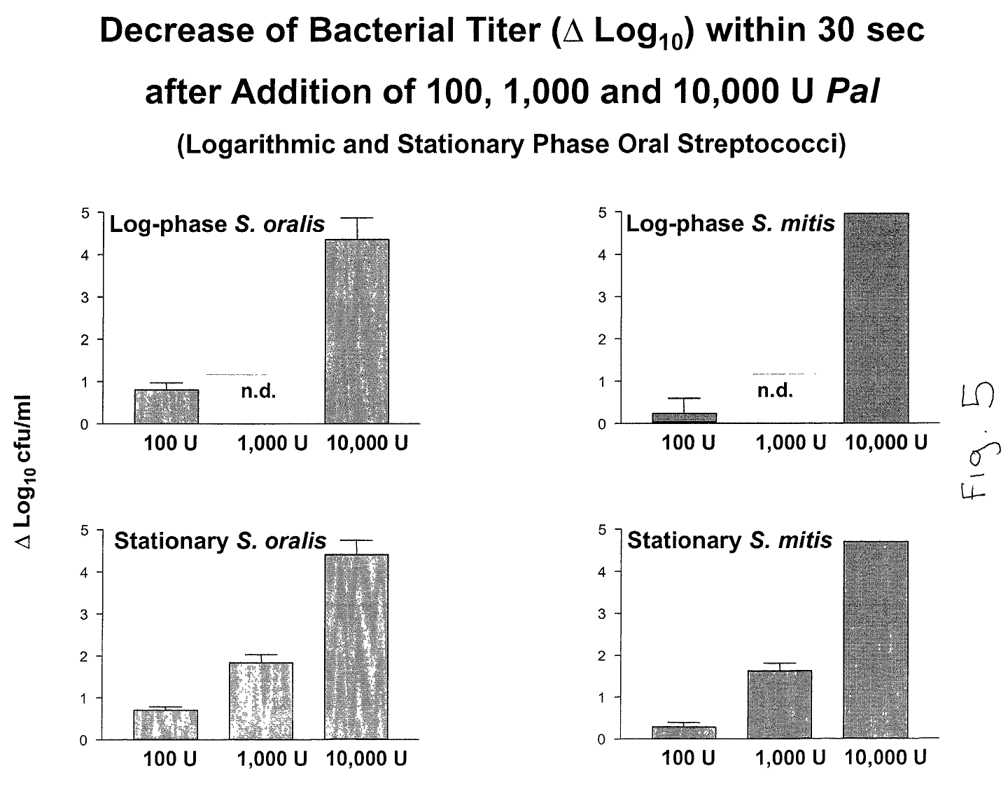

Streptococci oralis and Streptococci.mitis in log or stationary phases of growth were treated with different concentrations of the Pal lytic enzyme. Viability was measured after 30 seconds. Results, as shown in FIG. 5, indicate that both bacterial species were equally sensitive to the Pal enzyme in both log or stationary phases of growth.

Many modifications and variations of the present invention are possible in light of the above teachings. It is, therefore, to be understood within the scope of the appended claims the invention may be protected otherwise than as specifically described.

Each publication cited herein is incorporated by reference in its entirety.

What we claim is:

1. A method for treating bacterial infections, comprising the steps:
a) obtaining a composition comprising an effective amount of at least one lytic enzyme; and
b) applying said composition to a site of the infection
wherein the enzyme is produced by infecting a bacterium causing said infection with a bacteriophage specific for said bacteria and wherein said bacteria produces said at least one recombinant lytic enzyme selected from the group consisting of chimeric lytic enzymes, shuffled lytic enzymes, and combinations thereof.

2. The method according to claim 1, wherein said method for treating bacterial infections is used for the prophylactic treatment of infections.

3. The method according to claim 1, wherein said method for treating bacterial infections is used for the therapeutic treatment of infections.

4. The method according to claim 1, wherein said method further comprises including at least one holin enzyme in said composition.

5. The method according to claim 4, wherein said at least one holin enzyme is selected from the group consisting of chimeric holin lytic enzymes and shuffled lytic enzymes.

6. The method according to claim 1, wherein said composition further comprises at least one antibiotic that potentiates the bactericidal activity of the lytic enzyme.

7. The method according to claim 1, comprising a first lytic enzyme that is recombinant and a second lytic enzyme that is not recombinant.

8. The method according to claim 1, further comprising delivering said at least one lytic enzyme in a carrier suitable for delivering said lytic enzyme to the site of the infection.

9. The method according to claim 1, wherein the bacterial infection is Hemophilus influenza.

10. A method according to claim 1, wherein the at least one lytic enzyme is active against a bacterium selected from the group consisting of Pseudomonas, Streptococcus pneumoniae, Streptococcus fasciae, Listeria, Salmonella, E. coli, Campylobacter, Helicobacter pylori, Pseudomonas. Streptococcus mutans, Mycobacterium tuberculosis and Streptococcus.

11. The method according to claim 1, wherein the carrier is selected from the group consisting of an inhalant, a topical cream, a nasal spray, a syrup, a tablet, tampon, a suppository, an eye drop solution, a candy, a chewing gum, a lozenge, a troche, a powder, an aerosol, a liquid, a liquid spray, a bandage, a toothpaste and an oral wash.

12 A method as described in claim 1, wherein the bacterial infection is an infection of the upper respiratory tract, and the carrier is suitable for delivery of said at least one lytic enzyme to a mouth, throat, or nasal passage.

13. The method according to claim 12, wherein said method is used for the prophylactic treatment of infection.

14. The method according to claim 12, wherein said method for treating bacterial infection is used for the therapeutic treatment of infection.

15. The method according to claim 12, wherein said method further comprises including at least one holin enzyme in said composition.

16. The method according to claim 15, wherein said holin enzyme is a shuffled holin enzyme and/or a chimeric holin enzyme.

17. The method according to claim 10, wherein said bacteria being treated is Streptococcus pneumoniae or Hemophilus influenza.

18. The method according to claim 10, wherein said carrier is selected from the group consisting of a candy, chewing gum, lozenge, troche, tablet, a powder, an aerosol, a liquid and a liquid spray.

19. The method according to claim 10, wherein said composition further comprises a buffer that maintains pH of the composition at a range between about 4.0 and about 9.0.

20. The method according to claim 19, wherein the buffer maintains the pH of the composition at the range between about 5.5 and about 7.5.

21. The method according to claim 10, wherein said composition comprises a substance selected from the group consisting of a reducing reagent, dithiothreitol, a metal chelating reagent, ethylenediaminetetracetic disodium salt, a citrate-phosphate buffer and a sweetener.

22. The method according to claim 1, wherein the at least one lytic enzyme is lyophilized.

23. The method according to claim 1, further comprising administering a concentration of about 100 to about 500,000 active enzyme units of the lytic enzyme per milliliter of fluid in the wet environment of the nasal or oral passages.

24. The method according to claim 23, further comprising administering the concentration of about 100 to about 1100,000 active enzyme units per milliliter of fluid in the wet environment of the nasal or oral passages.

25. The method according to claim 11, wherein the composition is used for the therapeutic or prophylactic treatment of Streptococcus infection.

26. The method according to claim 111, wherein the composition is used for the therapeutic or prophylactic treatment of Hemophilus infections.

27. A composition for the treatment of a bacterial infection of an upper respiratory tract, prepared by a process comprising the steps:
a) obtaining at least one lytic enzyme produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria; and
b) admixing said at least one lytic enzyme with a carrier suitable for delivery to a mouth, throat, or nasal passage.

28. A composition as described in claim 27, further comprising a holin lytic enzyme.

29. The composition according to claim 28, wherein said holin lytic enzyme is selected from the group consisting of shuffled holin lytic enzymes and chimeric holin lytic enzymes.

30. The composition according to claim 27, wherein said bacteria being treated is Streptococcus pneumoniae or Hemophilus influenza.

31. The composition according to claim 27, wherein said carrier is selected from the group consisting of a candy, chewing gum, lozenge, troche, tablet, a powder, an aerosol, a liquid and a liquid spray.

32. The composition according to claim 27, wherein said composition further comprises a buffer that maintains pH of the composition at a range between about 4.0 and about 9.0.

33. The composition according to claim 32, wherein the buffer maintains the pH of the composition at the range between 5.5 and 7.5.

34. The composition according to claim 27, further comprising a bactericidal or bacteriostatic agent as a preservative.

35. The composition according to claim 27, wherein said at least one lytic enzyme is lyophilized.

36. The composition according claim 35, wherein said at least one lytic enzyme is present in a concentration of about 100 to about 500,000 active enzyme units per milliliter of fluid in the wet environment of the nasal or oral passages.

37. The composition according to claim 36, wherein said at least one lytic enzyme is present in a concentration of about 100 to about 10,000 active enzyme units per milliliter of fluid in the wet environment of the nasal or oral passages.

38. A composition for the treatment of a bacterial infection of the digestive tract, prepared by a process comprising the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and a carrier for suitable for delivering said at least one lytic enzyme to said digestive tract.

39. The composition according to claim 38, further comprising a holin enzyme.

40. The composition according to claim 39, wherein said holin enzyme is a shuffled holin enzyme.

41. The composition according to claim 39, wherein said holin enzyme is a chimeric holin enzyme.

42. The composition according to claim 38, wherein said bacteria to be treated are selected from the group consisting of Listeria, Salmonella, E. coli, and Campylobacter.

43. The composition according to claim 38, wherein said carrier for delivering said at least one lytic enzyme to the digestive tract is selected from the group consisting of suppository enemas, syrups, or enteric coated pills.

44. The composition according to claim 38, wherein said composition further comprises a buffer that maintains pH of the composition at a range between about 4.0 and 9.0.

45. The composition according claim 38, wherein said at least one modified lytic enzyme is present in a concentration of about 100 to about 100,000 active enzyme units per milliliter of fluid in the wet environment of the digestive tract.

46. The composition according to claim 45, wherein said at least one modified lytic enzyme is present in a concentration of about 100 to about 10,000 active enzyme units per milliliter of fluid in the wet environment of the digestive tract.

47. A composition for the therapeutic or prophylactic treatment of bacterial infections of burns and wounds of the skin, comprising:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and a carrier for suitable for delivering said at least one lytic enzyme to the skin.

48. The composition according to claim 47, further comprising a holin enzyme.

49. The composition according to claim 48, wherein said holin enzyme is a shuffled holin enzyme.

50. The composition according to claim 48, wherein said holin enzyme is a chimeric holin enzyme.

51. The composition according to claim 47, wherein said carrier is a bandage.

52. The composition according to claim 47, further comprising using said composition in the prophylactic treatment of bacterial infections.

53. The composition according to claim 47, further comprising using said composition in the therapeutic treatment of bacterial infections.

54. The composition according to claim 47, wherein said bacteria being treated is Pseudomonas.

55. The composition according to claim 47, wherein said bacteria being treated is Staphylococcus.

56. The composition according to claim 47, wherein said bacterium being treated are Staphylococcus and Pseudomonas.

57. A method for the therapeutic or prophylactic treatment of bacterial infections of burns and wounds of the skin, comprising:
a) obtaining a composition comprising an effective amount of at least one lytic enzyme, wherein said composition is prepared by the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and
b) applying said composition to a site of the burns and wounds of the skin.

58. The method according to claim 57, further comprising a holin enzyme.

59. The method according to claim 58, wherein said holin enzyme is a shuffled holin enzyme.

60. The method according to claim 58, wherein said holin enzyme is a chimeric holin enzyme.

61. The method according to claim 57, wherein said carrier is a bandage.

62. The method according to claim 57, further comprising using said composition in the prophylactic treatment of bacterial infections.

63. The method according to claim 57, further comprising using said composition in the therapeutic treatment of bacterial infections.

64. The method according to claim 57, wherein said bacteria being treated is Pseudomonas.

65. The method according to claim 57, wherein said bacteria being treated is Staphylococcus.

66. The method according to claim 57, wherein said bacteria being treated are Staphylococcus and Pseudomonas.

67. The method according to claim 57, further comprising at least one lytic enzyme which is not a enzyme.

68. A method for the prophylactic and therapeutic treatment of vaginal infections, comprising:
obtaining a composition comprising an effective amount of at least one lytic enzyme, wherein said composition is prepared by the steps of:
a) obtaining a composition comprising an effective amount of at least one lytic enzyme, wherein said composition is prepared by the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and
b) applying said composition to the vagina.

69. The method according to claim 68, further comprising administering a holin enzyme with said at least one lytic enzyme.

70. The method according to claim 69, wherein said holin enzyme is a shuffled holin enzyme.

71. The method according to claim 69, wherein said holin enzyme is a chimeric holin enzyme.

72. The method according to claim 68, wherein said carrier is to be placed in the vagina.

73. The method according to claim 68, wherein said carrier is a tampon.

74. The method according to claim 68, wherein said carrier is a pad.

75. The method according to claim 68, wherein said carrier is a douche.

76. The method according to claim 68, wherein said at least one lytic enzyme is specific for Group B Streptococcus.

77. A composition for the treatment of bacterial infection of a vagina, prepared by a process comprising the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and a carrier for suitable for delivering said at least one lytic enzyme to said vagina.

78. The composition according to claim 77, further comprising administering a holin enzyme with said at least one lytic enzyme.

79. The composition according to claim 78, wherein said holin enzyme is a shuffled holin enzyme.

80. The composition according to claim 77, wherein said holin enzyme is a chimeric holin enzyme.

81. The composition according to claim 77, wherein said carrier is a tampon.

82. The composition according to claim 77, wherein said carrier is a douche.

83. The composition according to claim 77, wherein said carrier is a pad.

84. The composition according to claim 77, wherein said lytic enzyme is specific for Group B Streptococcus.

85. The composition according to claim 77, further comprising a lytic enzyme which is not a lytic enzyme.

86. A method for treating bacterial infections of an eye comprising the steps of:
a) obtaining a composition comprising an effective amount of at least one lytic enzyme, wherein said composition is prepared by the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and a carrier for suitable for delivering said at least one lytic enzyme to said eye, and
b) applying said composition to said eye.

87. The method according to claim 86, further comprising administering a holin enzyme with said at least one lytic enzyme.

88. The method according to claim 87, wherein said holin enzyme is a shuffled holin enzyme.

89. The method according to claim 87, wherein said holin enzyme is a chimeric holin enzyme.

90. The method according to claim 86, wherein said bacteria being treated is Hemophilus.

91. The method according to claim 86, wherein said bacteria being treated is Staphylococcus.

92. The method according to claim 86, wherein the carrier is an eye drop solution.

93. The method according to claim 86, wherein the carrier is an eye wash solution.

94. The method according to claim 86, wherein said solution is an isotonic solution.

95. A composition for the treatment of a bacterial infection of the digestive tract, prepared by a process comprising the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and a carrier suitable for delivering said at least one lytic enzyme to said digestive tract.

96. The composition according to claim 95, further comprising a holin enzyme.

97. The composition according to claim 96, wherein said holin enzyme is a shuffled holin enzyme.

98. The composition according to claim 97, wherein said holin enzyme is a chimeric holin enzyme.

99. The composition according to claim 95, wherein said bacteria being treated is Hemophilus.

100. The composition according to claim 95, wherein said bacteria being treated is Staphylococcus.

101. The composition according to claim 95, wherein said carrier is an isotonic solution.

102. The composition according to claim 101, wherein said isotonic solution is in an eye drop dispenser.

103. A method for the prophylactic or therapeutic treatment of dermatological infections comprising:
a) obtaining a composition comprising an effective amount of at least one lytic enzyme, wherein said composition is prepared by the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell wall of said bacteria, and
2) admixing said at least one lytic enzyme with a carrier suitable for delivery of said at least one lytic enzyme, and
b) topically applying said composition to the skin.

104. The method according to claim 103, further comprising administering at least one holin enzyme.

105. The method according to claim 104, wherein said at least one holin enzyme is a shuffled holin enzyme.

106. The method according to claim 104, wherein said at least one holin enzyme is a chimeric holin enzyme.

107. The method according to claim 103, wherein said carrier is selected from the group consisting of an aqueous liquid, an alcohol base, a water soluble gel, a lotion, an ointment, a nonaqueous liquid base, a mineral oil base, a blend of mineral oil and petrolatum, lanolin, liposomes, hydrophilic gelling agents, cross-linked acrylic acid polymers (carbomer), cellulose polymers, hydroxy ethyl cellulose, cellulose gum, MVE/MA decadiene crosspolymers, PVM/MA copolymers, and any combinations thereof.

108. The method according to claim 103, wherein the form in which the composition is delivered is selected from the group consisting of a spray, a smear, a time release patch, a liquid absorbed wipe, and any combinations thereof.

109. The method according to claim 103, wherein said at least one lytic enzyme is in an environment having a pH which allows for activity of said lysin enzyme.

110. The method according to claim 109, wherein said composition further comprises a buffer that maintains pH of the composition at a range between about 4.0 and about 9.0.

111. The method according to claim 103, wherein said composition further comprises a mild surfactant in an amount effective to potentiate effects of the lytic enzyme.

112. The method according to claim 103, wherein the composition further comprises at least one complementary agent which potentiates the bactericidal activity of the lytic enzyme, said complementary agent being selected from the group consisting of penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate and chelating agents in an amount effective to synergistically enhance effects of the lytic enzyme.

113. The method according to claim 103, wherein the composition further comprises lysostaphin for the treatment of any Staphylococcus aureus bacteria.

114. The method according to claim 103, wherein said at least one lytic enzyme is present in an amount ranging from about 100 to about 500,000 units per milliliter.

115. A composition for the treatment of bacterial infections of the mouth or teeth, comprising prepared by a process comprising the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for delivery of said at least one lytic enzyme, and a carrier for suitable for delivering said at least one lytic enzyme to said mouth or teeth.

116. The composition according to claim 115, further comprising administering a holin enzyme.

117. The method according to claim 116, wherein said holin enzyme is a shuffled holin enzyme.

118. The method according to claim 116, wherein said holin enzyme is a chimeric holin enzyme.

119. The composition according to claim 115, wherein said composition is used for the prophylactic treatment of dental caries.

120. The composition according to claim 115, wherein said composition is used for the therapeutic treatment of dental caries.

121. The composition according to claim 115, wherein said carrier is selected from the group consisting of a toothpaste, an oral wash, a chewing gum and a lozenge.

122. The composition according to claim 115, wherein said bacteria being treated is Streptococcus mutans.

123. The composition according to claim 115, wherein said lytic enzyme is present in an amount ranging from about 100 to about 500,000 units per milliliter.

124. The composition according to claim 123, wherein said lytic enzyme is present in an amount ranging from about 10,000 to about 100,000 units per milliliter.

125. A method for parenterally treating bacterial infections, comprising the steps of:
a) obtaining a composition comprising an effective amount of at least one lytic enzyme, wherein said composition is prepared by the steps of:
1) obtaining at least one lytic enzyme wherein said at least one lytic enzyme is produced by infecting a bacteria causing said bacterial infection with a bacteriophage specific for said bacteria wherein said bacteria produces said at least one lytic enzyme selected from the group consisting of chimeric lytic enzymes, shuffled lytic enzymes, and combinations thereof, and wherein said at least one lytic enzyme has the ability to digest a cell of said bacteria, and
2) admixing said at least one lytic enzyme with a carrier suitable for parenterally delivering said at least one lytic enzyme, and
b) parenterally administering said composition to a site of the infection.

126. The method according to claim 125, wherein said method for treating bacterial infections is used for the prophylactic treatment of infections.

127. The method according to claim 125, wherein said method for treating bacterial infections is used for the therapeutic treatment of infections.

128. The method according to claim 125, further comprising at least one holin lytic enzyme.

129. The method according to claim 128, wherein said holin enzyme is a chimeric holin enzyme.

130. The method according to claim 128, wherein said holin enzyme is a shuffled holin enzyme.

131. The method according to claim 125, further comprising at least one lytic enzyme which is not a lytic enzyme.

132. The method according to claim 125, wherein the at least one lytic enzyme is for the treatment of Pseudomonas.

133. The method according to claim 125, wherein the at least one lytic enzyme is for the treatment of Streptococcus.

134. The method according to claim 125, wherein the at least one lytic enzyme is for the treatment of Staphylococcus.

135. The method according to claim 125, wherein the at least one lytic enzyme is for the treatment of Clostridium.

136. The method according to claim 125, wherein said composition further comprises a buffer that maintains pH of the composition at a range between about 4.0 and about 9.0.

137. The method according to claim 136, wherein the buffer maintains the pH of the composition at the range between about 5.5 and about 7.5.

138. The method according to claim 136, wherein said composition comprises an agent selected from the group consisting of a reducing reagent, dithiothreitol, a metal chelating reagent, ethylenediaminetetracetic disodium salt, a citrate-phosphate buffer, and a bactericidal or bacteriostatic preservative.

139. The method according to claim 125, wherein said at least one lytic enzyme is lyophilized.

140. The method according to claim 125, wherein said composition is administered intravenously, intramuscularly or subcutaneously.

141. The method according to claim 125, wherein said composition further comprises at least one complementary agent which potentiates the bactericidal activity of the lysine enzyme, said complementary agent being selected from the group consisting of penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate and chelating agents in an amount effective to synergistically enhance the therapeutic effect of the lysin enzyme.

142. The method according to claim 125, wherein said carrier comprises of distilled water, a saline solution, albumin, a serum, and any combinations thereof.

143. The method according to claim 125, wherein said carrier further comprises preservatives.

144. The method according to claim 143, wherein said preservatives comprise p-hydroxybenzoates.

145. The method according to claim 125, wherein said carrier comprises an isotonic solution for an injection, said isotonic solution comprising a compound selected from group consisting of sodium chloride, dextrose, mannitol, sorbitol, lactose, phosphate buffered saline, gelatin, albumin, a vasoconstriction agent and combinations.

146. The method according to claim 125, wherein said further carrier further comprises DMSO.

147. The composition according to claim 1, wherein said method for treating bacterial infections is used for the prophylactic treatment of infections.

148. The composition according to claim 1, wherein said method for treating bacterial infections is used for the therapeutic treatment of infections.

149. The composition according to claim 1, wherein said at least one holin lytic enzyme is a shuffled holin lytic enzyme.

150. The composition according to claim 1, wherein said holin enzyme is a chimeric holin lytic enzyme.

151. The composition according to claim 1, further comprising at least one lytic enzyme which is not selected from the group consisting of at least one shuffled lytic enzyme, chimeric lytic enzyme, and holin lytic enzyme.
